(19) United States
(12) Patent Application Publication
(10) Pub. No.: US 2024/0407915 A1
(43) Pub. Date: Dec. 12, 2024

(54) One Size Expandable Heart Valve

(71) Applicants: Krishna Raj KUPUMBATI, Irvine, CA (US); Ria Shree KUPUMBATI, Irvine, CA (US)

(72) Inventors: Krishna Raj KUPUMBATI, Irvine, CA (US); Ria Shree KUPUMBATI, Irvine, CA (US)

(21) Appl. No.: 18/676,869

(22) Filed: May 29, 2024

Related U.S. Application Data
(60) Provisional application No. 63/471,959, filed on Jun. 8, 2023.

(51) Int. Cl. A61F 2/24

(52) U.S. Cl. CPC A61F 2/2418; A61F 2/2433; A61F 2/2436

(57) ABSTRACT

A transcatheter stent valve for heart valve replacement having a stent frame formed from a SE portion extending throughout the length of the stent frame and a BE portion extending lengthwise throughout and connected together in series around the circumference of the stent frame. The SE portion provides outward force against the annulus of the native heart tissues, the BE portion allows the perimeter to be adjusted to accommodate a large range of heart valve perimeters.

### BACKGROUND

In the current transcatheter heart valve industry, multiple size valves are manufactured to meet different annulus sizes of the patient. Even with these multiple sizes, it can be challenging to get the valve size to match exactly to the patient annulus size. There is a need for a valve that can custom fit the patient's annulus size. Current transcatheter heart valves have an expandable stent or expandable metal cage that is attached to and provide support for bioprosthetic/synthetic leaflets to make the heart valve. Some of such stents are self-expanding (SE) and expand outwards via stored elastic energy to make contact with the valve annulus. These SE stents provide an outward force onto the annulus that cannot be too low in order to prevent stent migration relative to the annulus. The outward force of the SE stent cannot be too large or else the stent can grow into and through the vessel wall. Balloon expandable (BE) stents have a specified size range (i.e., effective diameter range) that they are intended for; over expansion can hurt the annulus and cause dissection; under expansion often leads to perivalvular leaks around the stent.

What is needed is a stent that can accommodate a wide range of sizes for a heart annulus. The stent should provide a minimum of outward force onto the annulus to ensure that stent migration and perivalvular leakage is minimized. The stent should support bioprosthetic leaflets in a manner that does not compromise the durability of the leaflets.

### SUMMARY

The present invention is a stent valve formed from a stent frame that can be made custom fit to each patient's size annulus of the heart. The stent frame provides the support for replacement tissue leaflets such that the stent valve of the present invention is delivered to the annulus of the heart via transcatheter methods. The stent frame consists of SE portions and BE portions that are located in series around the perimeter of the stent frame; the SE portions and BE portions extend throughout the entire axial length of the stent frame. The SE portions are made up of shape memory material such as Nitinol, elastomeric materials, or expandable/collapsible polymers that expand outwards via stored energy within the stent. The BE portions are formed from a stent material that is plastically deformable such as cobalt chromium, stainless steel or other material used in BE stents that can be expanded to the required size via a dilation balloon.

In one embodiment the stent frame has three SE portions and three BE portions, for example, positioned in series around the perimeter of the stent (the stent may contain more or less than six portions). The SE and BE portions are assembled together using sutures, for example, with the SE portions in the expanded configuration (i.e., the equilibrium configuration) and the BE portions in a crimped configuration at room temperature and at body temperatures; other methods of attachment are anticipated as have been used in the medical device industry for attachment. During the assembly of the stent valve, when the SE portions and BE portions are being connected together in series, the leaflets are assembled onto the stent. Three leaflet commissural pads (for a three leaflet valve, for example) can be attached to the BE portions of the stent frame at or near the downstream end of the stent frame.

Prior to implantation, the stent can be loaded on the desired dilation balloon of a delivery catheter and the combined dilation balloon and stent can be confined within a delivery sheath having a delivery profile of 20 French (range 18-30 French).

Once the stent valve has been delivered to the annulus, the stent frame can be expanded to the desired annulus size and implanted. During implantation, the delivery sheath is removed and the SE portion expands outwards to its equilibrium configuration (resembling the room temperature shape) and the BE portions can then be expanded via a dilation balloon to fill the necessary annular space.

This stent valve can be implanted into a patients' heart as a replacement heart valve and can serve several purposes: Depending on the patient's annulus size the valve can be balloon expanded to custom fit the patient (one valve can fit multiple annulus sizes); additionally, this valve can be implanted in growing children, and it can be expanded via a dilation balloon in a separate procedure as children grow (one valve for the children during growth years).

The stent valve of the present invention can include expansion limiters in the SE portions that limit the amount of expansion that the SE portion can undergo. As the stent frame is being further expanded (i.e., following the SE expansion step, for example) by a dilation balloon, the expansion limiters can retain the SE portions in a configuration that provides a minimal outward force onto the annulus and requires the BE portions to expand outwards to increase the perimeter of the stent frame. The result is a stent frame that provides at least a minimal amount of outward force to reduce the likelihood of stent frame migration and prevent perivalvular leaks, and allows the BE portions to be expanded outwards as required to meet the variable diameters of the annulus in a variety of patients using one size of the stent frame of the present invention.

The stent frame of the present invention provides benefits for use as a replacement stent valve for one of the valves of the heart, however, it is understood that the stent frame of the present invention can also be used without replacement leaflets to provide support for blood vessels of the body or for support for other chambers within the body. The stent structure for these other applications is the same as described for the stent valve with SE portions attached in series to BE portions along the perimeter of the stent.

### BRIEF DESCRIPTION OF THE DRAWINGS

FIG. 1A is a plan view of a splayed out stent frame comprised of three SE portions and three BE portions connected together in series in a circumferential direction with the BE portions in a crimped configuration and the SE portion being expanded to an equilibrium configuration.

FIG. 1B is a top view of the stent valve showing three SE portions in series with three BE portions and having 3 leaflets attached to the stent frame.

FIG. 1C is a perspective view of the stent valve showing a zig zag stent structure formed from three BE portions connected in series with three SE portions.

FIG. 2 is a perspective view of the stent frame positioned over a dilation balloon and contained in a small diameter delivery configuration within a sheath.

FIG. 3 is a perspective view of the stent frame after it has been released from a sheath and the SE portion expanded outwards and the BE portion has remained in a crimped configuration; the stent frame has not made contact with the annulus and being held in position by a coiled guidewire.

FIG. 4A is a plan view of a splayed out stent frame showing expansion of both the SE portion and also the BE portion after a dilation balloon has been inflated.

FIG. 4B is a perspective view of the stent frame being expanded by a dilation balloon to expand the BE portion outwards.

FIG. 4C is a perspective view of the stent frame after the dilation balloon has been deflated and the BE portion and SE portion of the stent frame have rebounded to a smaller diameter.

FIG. 5A shows an expansion limiter extending across SE members of the SE portion of the stent frame having a zig zag open structure thereby allowing the BE portion to expand to a greater extent during inflation of the balloon.

FIG. 5B shows an expansion limiter extending across SE members of the SE portion of the stent frame having a closed structure thereby allowing the BE portion to expand to a greater extent during inflation of the balloon.

FIG. 6A is a semi-perspective view of the stent frame having leaflets attached and the leaflets having additional coaptation distance to provide for maintaining coaptation if the stent frame was enlarged to a larger diameter.

FIG. 6B is a semi-perspective view of the stent frame having leaflets attached and the leaflets maintaining coaptation distance after the stent frame has been enlarged to a larger diameter.

### DETAILED DESCRIPTION

This specification will focus on a stent valve embodiment for use as a replacement heart valve, but it is understood that the stent frame that is described can equally be used as a stent for vascular applications; the stent frame used for vascular applications can be formed without the presence of replacement leaflets.

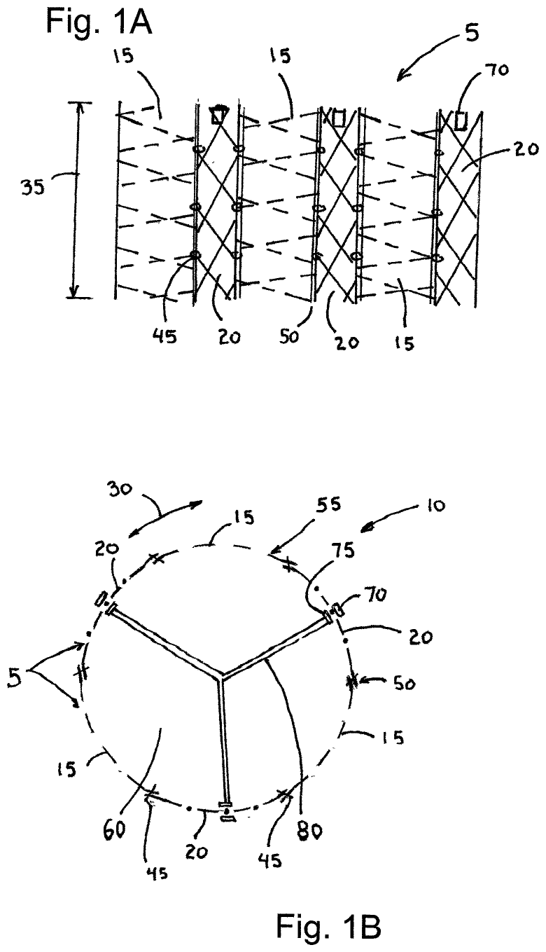

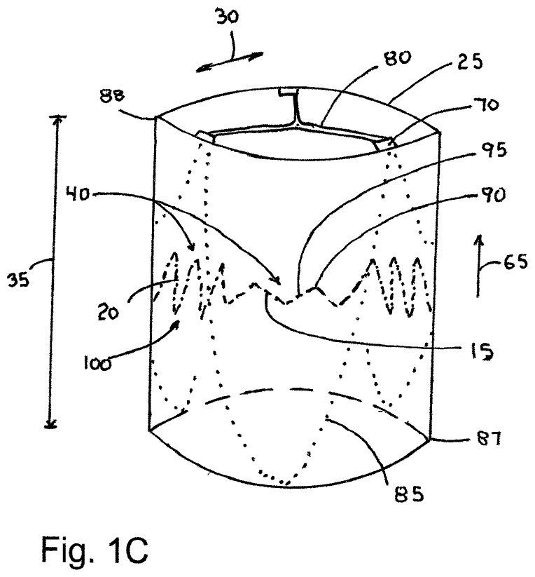

FIGS. 1A-1C show the stent frame 5 and stent valve 10 in an assembly configuration. FIG. 1A shows an embodiment of the present invention with three SE portions 15 and three BE portions 20 of a splayed-open and flattened stent frame 5. The SE portions 15 are positioned in series with the BE portions 20 along the stent frame perimeter 25 in a circumferential direction 30 and extend throughout the axial length 35 of the stent frame 5. The stent structure 40 of the SE portion 15 or the BE portion 20 can be a braided structure, a laser machined structure, a wire structure or other stent structure 40 found in expandable stents used in the body. The stent structure 40 can have an open pattern for the stent struts such as a zig zag structure or it can be a closed structure such as those that can be formed from laser machining, for example. The SE portions 15 are connected to the BE portions 20 forming frame connections 45 using sutures, fibers, adhesives, or other mechanical attachment members and using methods of connecting a SE stent portion to a BE stent portion that do not lead to stent fracture failure at the frame connections 45. The SE portion 15 can be formed of Nitinol or other elastomeric metal or elastomeric polymer; the BE portion 20 can be formed from stainless steel, cobalt chromium material, or other BE material used in BE stents. The BE portion 20 can be overlapped with the SE portion 15 by 2 mm (range 1-5 mm) forming an overlap region 50 to maintain a uniform curvature to the stent frame surface 55 of the frame connection as it is being expanded and implanted adjacent to the heart annulus. Other methods of forming the frame connection between a Nitinol material and a stainless steel metal, for example, using thermal methods or machining methods can often lead to stent fracture failure at the frame connections 45 due to flexation of the stent.

Leaflets 60 attach to the stent frame 5 to direct blood flow in a downstream direction 65. Near the downstream end of the BE portions 20 are located commissural attachment sites 70 which will be used to attach leaflet commissural pads 75 as shown in FIG. 1B. FIG. 1B shows a top view of the downstream end of the stent frame 5 with three SE portions 15 forming frame connections 45 to three BE portions 20 in series along the perimeter 25 of the stent frame 5 (see FIG. 1C). Three leaflets 60 are shown with their free edges 80 coapting with a free edge 80 of a neighboring leaflet 60 FIG. 1C shows a perspective view of the stent valve 10 with leaflets 60 attached to the stent frame 5 along a crown-shaped attachment path 85 extending from an upstream end 87 of the stent frame 5 to a downstream end 88. The stent structure 40 (i.e., its hinge 90 or bend regions and stent struts 95) can be an open zig zag structure as shown in only a portion of the stent frame for clarity; the stent structure 40 in this embodiment would extend throughout the entire axial length 35 of the stent frame 5 but is shown in only a portion of the axial length 35 for demonstration purposes. Other stent structures 40 including closed cell structures are anticipated. The stent structure 40 of FIG. 1C is shown in its initial equilibrium configuration with the SE portion 15 expanded outwards with the stent struts 95 oriented substantially in a circumferential direction 30 and the BE portion 20 in its crimped configuration 100.

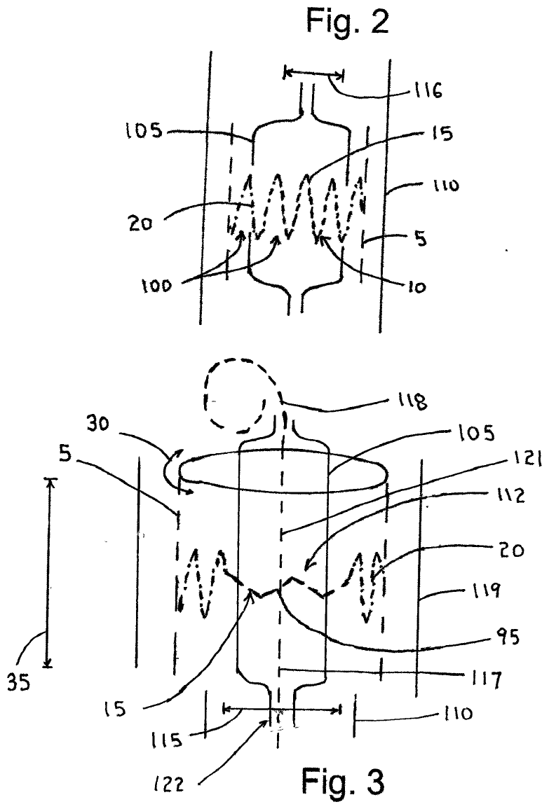

FIG. 2 shows the stent valve 10 positioned over an uninflated dilation balloon 105 and being held in a constrained configuration within a sheath 110 for transcatheter delivery to the annulus of the heart. A portion of the stent structure 40 along the axial length 35 is shown with both the BE portion 20 and the SE portion 15 being held in a crimped configuration 100 with a SE portion circumferential dimension 116 with the stent struts 95 being held in tight approximation to a neighboring stent strut.

After release from the sheath 110 the SE portions 15 are able to expand outwards to an equilibrium configuration and the BE portions remain in a crimped configuration as shown in the spayed out view in FIG. 1A. The BE portions 20 have not yet been expanded via the dilation balloon 105. FIG. 3 shows a perspective view of the stent frame after it has been released from the sheath; only a portion of a zig zag stent structure, for example, is shown along the axial length 35 of the stent frame 5, although it is understood that the stent structure extends along the entire axial length 35. The SE portions 15 have been expanded outwards to a SE portion equilibrium configuration 112 with an equilibrium expansion distance 115 that is greater than a SE portion circumferential distance 116 in its crimped configuration shown in FIG. 2. The stent struts 95 are oriented substantially in a circumferential direction 30 in their equilibrium configuration (i.e., in an open space or free space) such that further expansion of the SE portion 15 is not significantly achievable (due to geometrical considerations for circumferential direction of stent struts) and the SE stent structure 40 is incapable of significant further expansion (i.e., less than 10 percent further expansion with the outward force that is required to expand the BE portion 20 of 5 atm (range 3-15 atm).

In one embodiment of the present invention, the stent frame 5 is configured to make contact with the annulus 119 upon release from the sheath 110. Further expansion of the stent frame via the dilation balloon 105 to expand the BE portions 20 may not be required by the operator in this case. In an alternate embodiment, the stent frame 5 does not make initial contact with the annulus upon release from the sheath 5 as shown in FIG. 3. In this instance it can be necessary to ensure that the stent frame 5 does not migrate from its intended implantation site adjacent to the annulus, for example. To provide proper positioning of the stent frame 5 a guidewire 117 having a coil 118 located at its distal end is positioned within the dilation catheter 122 with the coil 118 positioned adjacent and distal to the stent frame 5. The coil 118 is configured to prevent the stent frame 5 from migrating distally via direct contact with the stent frame 5. Inflation of the dilation balloon 105 will then cause the BE portions 20 of the stent frame 5 to expand and place the stent frame 5 into contact with the annulus 119 providing a leak-tight seal with the annulus 119 and structural support to the annulus 119 and heart tissues as needed.

The guidewire can be formed from metal, polymeric material, or a composite material including Nitinol and polymeric coated guidewires. The coil 118 can be contiguous with the guidewire body 121 or alternately the coil 118 can be attached to the guidewire body 121 at the distal end of the guidewire 117 via a variety of bonding methods including welding, thermal bonding, adhesive bonding, mechanical attachment or other methods used in the medical device industry. The coil 118 has a greater flexibility than the guidewire body 121 to provide a less traumatic contact with the tissues of the heart.

Other structural members other than a coiled guidewire are anticipated to prevent migration of the stent frame 5 following removal of the sheath 110. For example, a fiber can extend within the sheath and form a releasable loop with the stent frame 5. The fiber holds the stent frame 5 from migrating upon release from the sheath 110. The loop can be released following dilation of the stent frame 5 via the dilation balloon 105. Other migration control members have been used in the medical device industry to prevent migration of a stent prior to balloon expansion. As another example, elastic wing members attached to a balloon dilation catheter near each end of the dilation balloon can hold each end of the stent frame 5 until the stent frame 5 has been fully expanded to a specified diameter. The elastic wing members then are forced to release the stent frame 5 when the stent frame 5 has been expanded via a dilation balloon to the specified diameter.

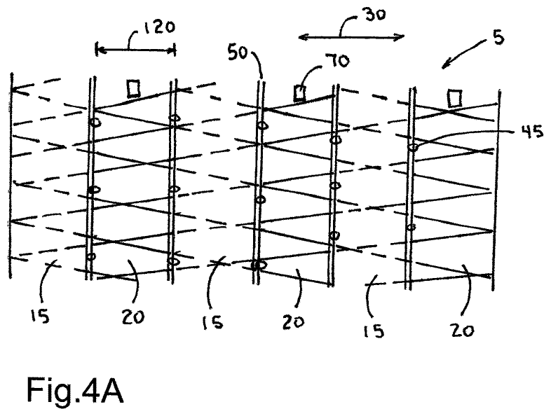

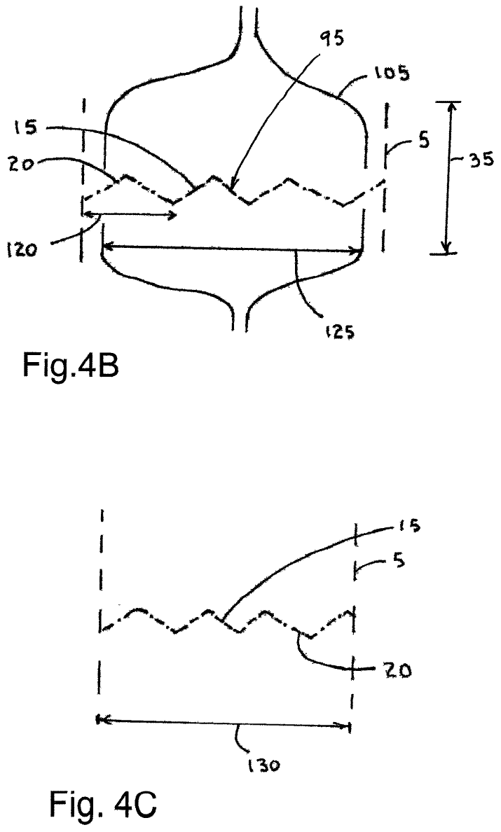

FIGS. 4A and 4B show the stent frame 5 after the dilation balloon 105 has expanded the BE portions 20. In FIG. 4A a splayed out stent frame 5 shows that the BE portion 20 have been enlarged in a circumferential direction 30 to a greater dimension than prior to balloon expansion. FIG. 4B shows the stent structure 40 along a portion of the stent axial length 35 having an expanded BE portions circumferential dimension 120 that is greater than the BE portion circumferential dimension in a crimped configuration. The stent frame is expanded into contact with native tissues of the heart including contact along a perimeter of the annulus of the heart. As the BE portions 20 are being expanded by the dilation balloon 105, some additional expansion is also expected for the SE portions 15 due to the balloon dilation. The SE portions 15 can be designed such that their orientation in their equilibrium configuration is directed with substantial circumferential direction 30 (i.e., the SE portion 15 can only expand an additional 10 percent or less due to the outward forces of the dilation balloon 105 of 5 atm (range 3-15 atm). Hence, upon inflation of the dilation balloon 105, very little further expansion of the SE portion 15 can be obtained and the expansion of the BE portions 20 will therefore dominate the frame expansion; expansion of the SE portion is restricted from further expansion due to the circumferential direction of the stent struts 95 of the SE portion 15. The dilation balloon 105 can dilate the annulus at a dilation 125 to 15 percent above the annulus native diameter (or annulus perimeter) to ensure that the SE portion 15 still maintains an outward force against the annulus following rebound of the stent frame 5, for example. FIG. 4C shows that some rebound of the stent frame 5 including rebound of the SE portion 15 can be expected upon deflation of the dilation balloon 105 where the stent frame diameter 130 is 5 percent less than the diameter of the stent frame 5 or of the dilation balloon 105 during expansion (range 0-15 percent less).

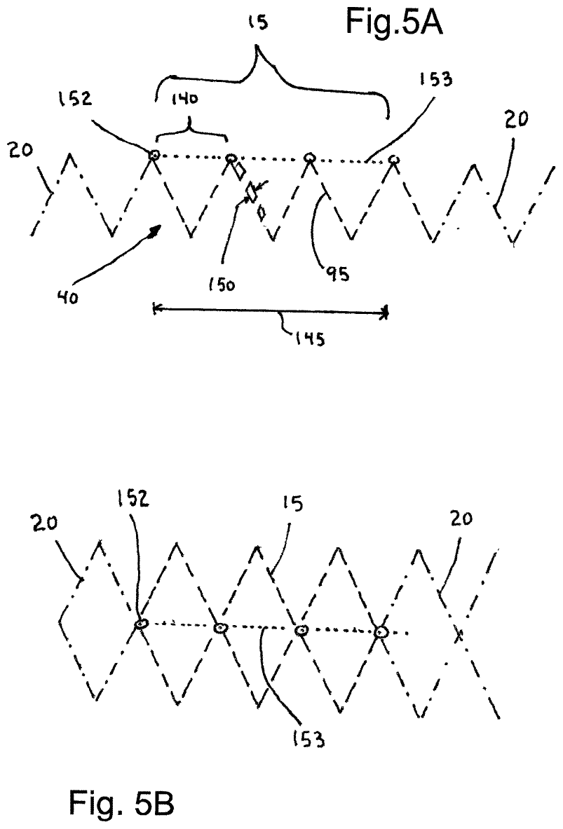

FIGS. 5A and 5B show an expansion limiter 135 that is a fiber or thin flexible member that extends across a SE member 140 (i.e., a SE strut and hinge) of the stent structure 40 and prevents or restricts the expansion of a SE portion 15 beyond a specified amount of controlled SE portion expansion distance 145. The specified amount of controlled expansion of the SE struts 95 or SE members of the SE portion 15 can be equal to expansion distance found in the configuration of the SE stent structure 40 when it has achieved its SE portion equilibrium expansion distance 115 (see FIG. 3B) in an open space or free space. In this case the expansion limiter 135 can be machined into the stent structure 40 of the SE portion 15 of the stent frame 5 with the stent frame 5 in its equilibrium state or equilibrium configuration 112. The expansion limiter 135 can be formed from the same material as the stent struts 95 and can be contiguous with the stent struts 95 but with a greater flexibility and smaller expansion limiter width than the stent strut width 150 along a stent frame surface 55 (see FIG. 1B).

Alternately, it can be preferable to ensure that a minimum amount of outward force is being applied by the SE portion 15 onto the annulus, for example, following expansion of the BE portion 20 of the stent frame 5 via the dilation balloon 105. In one embodiment, the expansion limiter 135 holds the SE stent structure 40 inwards to a SE portion expansion distance 145 that is less than the SE portion equilibrium expansion distance 115 (see FIG. 3B) and the SE portion 15 can exert a force outwards that is equivalent to a dilation balloon 105 outward force of 3 atmospheres (range 1-7 atm) following expansion of the stent frame 5 via the dilation balloon 105 (see FIG. 4B) to expand the BE portion 20.

As seen in FIG. 5A an open zig zag stent structure 40 can have loop features 152 formed within the stent material using laser machining, for example, such that a fiber (metallic or polymeric expansion limiter 135) can be placed into the loop feature 152 and tied or bonded to the loop feature 152 such that the SE portion 15 can only expand outwards until the expansion limiter 135 is taught or straight; further expansion of the SE portion is thereafter restricted by the expansion limiter. The SE portion 15 at this point can still provide an outward force equal to the specified outward force against the expansion limiter 135. As the BE portion 20 of the stent frame 5 is further expanded after contacting the annulus along its entire perimeter, the specified outward force supplied by the SE portion will be applied onto the annulus to prevent migration and to prevent perivalvular leakage. FIG. 5B shows a closed cell stent structure 40 having a loop feature 152 formed into the stent structure 40. An expansion limiter 135 such as a limiting cable 152 passing through and attached to the loop features 152 can restrict the amount of outward expansion of the SE portion 15 but still maintain an outward force provided by the SE portion 15. As the BE portion 20 is expanded, the operator can visualize the limiting cable 152 for straightness and provide a small additional expansion to the BE portion 20 to ensure that the SE portion 15 will be providing the outward force against the annulus that has been specified upon deflation of the dilation balloon 105.

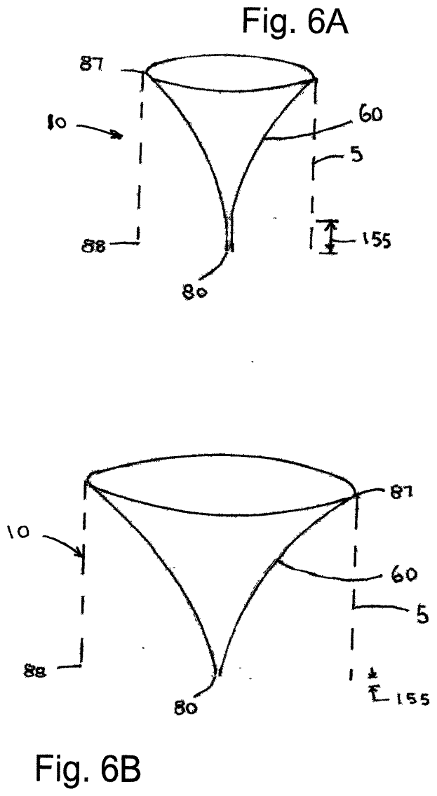

The replacement leaflets 60 for the present invention can have an additional amount of coaptation distance 155 for the leaflet free edges 80 as the stent valve 10 of the present invention is implanted as shown in FIG. 6A. Thus, when the stent frame 5 is expanded further by the operator via a dilation balloon 105, the leaflet free edges 80 will still coapt well as shown in FIG. 6B and not result in regurgitation or in unwanted wear of the leaflets 60 that can end in valve failure. The coaptation distance 155 of the replacement leaflets 60 can be at least 5 mm (range 5-15 mm).

Reference numerals that describe structure found in any figure in this specification can equally describe structure found in any other figure found in the specification.

1. A stent valve for transcatheter delivery and implantation into native tissues of the heart comprising,
A. a stent frame having one or more balloon expandable portions extending throughout an axial length of said stent frame,
B. said stent frame further having one or more self-expanding portions extending throughout said axial length of said stent frame,
C. said one or more balloon expandable portions being connected in series via frame connections with said one or more self-expanding portions in a circumferential direction around a perimeter of said stent frame,
D. said stent frame having replacement leaflets attached thereto to direct blood flow in a downstream direction.

2. The stent valve of claim 1 wherein said frame connections are formed from sutures, fibers, or other mechanical members.

3. The stent valve of claim 1 wherein said one or more self-expanding portions have a stent strut structure having a substantially circumferential direction that allows 10 percent or less further expansion of said one or more self-expanding portions.

4. The stent valve of claim 1 wherein said one or more balloon expandable portions are three balloon expandable portions and said one or more self-expanding portions are three self-expanding portions.

5. The stent valve of claim 1 wherein said one or more balloon expandable portions are two balloon expandable portions and said one or more self-expanding portions are two self-expanding portions.

6. The stent valve of claim 1 wherein said replacement leaflets have commissures that are attached to said one or more balloon expandable portions.

7. The stent valve of claim 1 wherein said replacement leaflets have free edges that have a coaptation distance with neighboring leaflet free edges, said coaptation distance being at least 5 mm.

8. The stent valve of claim 1 wherein said replacement leaflets have leaflet attachment edges that are attached to said one or more self-expanding portions and to said one or more balloon expandable portions along a crown-shaped attachment path.

9. The stent valve of claim wherein said one or more self-expanding portions have expansion limiters extending across self-expanding members of said one or more self-expanding portions to limit said self-expanding members to a controlled expansion distance.

10. The stent valve of claim 9 wherein said expansion limiters are formed contiguously with said stent frame of said one or more self-expanding portions.

11. The stent valve of claim 9 wherein said expansion limiters are formed from fibers that are attached to a loop feature in said stent structure of said one or more self-expanding portions.

12. The stent valve of claim 1 wherein said stent frame provides an outward force onto the native tissues of the heart of at least 3 atmospheres.

13. The stent valve of claim 1 wherein said stent frame is delivered on a dilation balloon with said stent frame being constrained by an outer sheath.

14. A stent for transcatheter delivery and implantation into native tissues of the body comprising,
A. a stent frame having one or more balloon expandable portions extending throughout an axial length of said stent frame,
B. said stent frame further having one or more self-expanding portions extending throughout said axial length of said stent frame,
C. said one or more balloon expandable portions being connected in series via frame connections with said one or more self-expanding portions in a circumferential direction around a perimeter of said stent frame.

15. The stent of claim 4 wherein said frame connections are formed from sutures, fibers, or other mechanical members.

16. The method of delivery of a stent valve for transcatheter delivery and implantation into native tissues of the heart comprising,
A. providing a stent frame having one or more balloon expandable portions extending throughout an axial length of said stent frame, said stent frame further having one or more self-expanding portions extending throughout said axial length of said stent frame, said one or more balloon expandable portions being connected in series to said one or more self-expanding portions along a perimeter of said stent frame, said stent frame having replacement leaflets attached thereto to direct blood flow in a downstream direction,
B. releasing said stent frame from a sheath to allow said one or more self-expanding portions to expand outwards,
C. inflating a dilation balloon to expand said one or more balloon expandable portions to place the stent frame into contact with the native tissues of the heart.

17. The method of claim 16 wherein said stent frame is further expanded via a dilation balloon to make contact with the native tissues of the heart that have been enlarged over time.

18. The method of claim 17 wherein said further expansion is due to expansion of said one or more balloon expandable portions, said one or more self-expanding portions having a stent strut structure that is restricted from further expansion.

19. The method of claim 18 wherein said restriction from further expansion of said one or more self-expanding portions is due to circumferential direction of stent struts in said one or more SE portions.

20. The method of claim 18 wherein said restriction from further expansion of said one or more self-expanding portions is due to expansion limiters extending across stent struts of said one or more self-expanding portions in an equilibrium configuration.

21. The method of claim 16 wherein prior to the step of inflating a dilation balloon, a coil located at the distal end of a guidewire is placed into contact with said stent frame to prevent migration of said stent frame.
